(19) United States
(12) Patent Application Publication
(10) Pub. No.: US 2026/0240512 A1
(43) Pub. Date: Aug. 20, 2026

(54) METHOD FOR HANDLING DRIFTING GEOMETRIC MISALIGNMENTS IN 3D X-RAY IMAGING

(71) Applicant: Siemens Healthineers AG, Forchheim (DE)

(72) Inventor: Frank DENNERLEIN, Eckental (DE)

(73) Assignee: Siemens Healthineers AG, Forchheim (DE)

(21) Appl. No.: 19/543,252

(22) Filed: Feb. 18, 2026

(30) Foreign Application Priority Data
Feb. 19, 2025 (EP) 25158830.7
Apr. 17, 2025 (EP) 25171139.6

(51) Int. Cl. A61B 6/00

(52) U.S. Cl. CPC A61B 6/5205; A61B 6/5288

(57) ABSTRACT

A computer-implemented method comprises: receiving a projection data set including a sequence of projection images and a sequence of geometry information, each geometry information being associated with a respective projection image; processing projection images, except the first projection image, iteratively image-by-image in order of the sequence; and reconstructing a 3D image data set based on the projection images and modified geometry information after the processing. The processing includes: calculating a cost function for the current projection image being processed depending on projection images and associated geometry information in the sequence up to and including the current projection image; modifying the geometry information associated with the current projection image to numerically minimize the cost function; and repeating the calculating and modifying the current projection image until a stopping criterium is met.

### CROSS-REFERENCE TO RELATED APPLICATION(S)

The present application claims priority under 35 U.S.C. § 119 to European Patent Application No. 25158830.7, filed Feb. 19, 2025, and European Application No. 25171139.6, filed Apr. 17, 2025, the entire contents of each of which is incorporated herein by reference.

### FIELD

The present disclosure relates to a computer-implemented method for handling drifting geometric misalignments in a projection data set of 3D X-ray imaging for reconstructing an image data set, a data processing unit, a 3D X-ray imaging system, a computer program product and a computer-readable medium.

### BACKGROUND

3D X-ray imaging is about generating a 3D image data set representing the density distribution of an object, in particular a patient, from a collection of a projection data set comprising projection images which are 2D X-ray images and which are acquired from different geometric points of view. This process is called tomographic reconstruction and is generally known.

Over the past decades, several mathematical formulas were derived that achieve such a reconstruction. Those mathematical formulas are the basis of the imaging reconstruction algorithms that are nowadays applied for 3D X-ray imaging. These algorithms generate a 3D image data set from the attenuation signals which are detected by the X-ray detector.

To achieve 3D image data sets of high image quality, it is ideal to know with high precision the geometric information of the points of view from which the projection images were acquired. Advantageously, the contributions of the individual projection images are aligned well to each other during the reconstruction process to generate a high image quality, in particular, sharp, 3D image data set. To the contrary, when the acquisition geometry and therefore the geometric information contains uncertainties, there could be geometric distortions, unsharp edges and/or double contours in the resulting 3D image data set. In this case, the image quality could be as low as the uncertainties render the 3D image data set unuseful.

Geometric uncertainties occur in practice, when a projection image is acquired from a different point of view than what is geometrically assumed. That is, its associated geometric information is not correct. Such a misalignment could be caused by an unprecise motion of the 3D X-ray imaging system and/or by an unknown motion of the object during acquisition.

In general, there are different types of misalignments. Some misalignments might be of global character, i.e., they are constant over the entire acquired sequence of projection images. Other misalignments are of local character, i.e., they differ independently from one projection image to another of the sequence. The latter ones are here called “geometric jitter”.

DE 10 2019 216 446 A1 discloses a computer-implemented method for determining the relative geometry of at least two projections acquired from different perspectives via transmission imaging of an object through which radiation is transmitted, the at least two projections acquired from different perspectives via transmission imaging are provided and the fundamental matrix linking at least two projections is determined at least approximately by solving an optimization problem taking into account a consistency condition existing between the Radon transforms of the at least two projections.

In “A New CT Rawdata Redundancy Measure applied to Automated Misalignment Correction”, The 12th International Meeting on Fully Three-Dimensional Image Reconstruction in Radiology and Nuclear Medicine, pages 264 to 267, C. Debbeler et al. publish a new rawdata redundancy measure. In computed tomography, redundantly measured rays (i.e. multiple measurements of a line integral through an object) pose a computationally efficient possibility to quantify the rawdata quality in a cost function and thus to reduce many kinds of artifacts. A general downside of such a cost function is that the portion of redundant rays is generally small and depends on the specific data acquisition geometry. The authors propose using information associated with plane integrals instead of line integrals in order to tremendously increase the rawdata utilization when formulating a cost function that quantifies the rawdata quality. In the cone-beam data acquisition geometry, which does not allow plane integrals to be measured directly, plane-information is obtained using the 2D Radon transform of the measured rawdata and a subsequent differentiation operation. The new rawdata redundancy measure is successfully applied for automatic misalignment correction.

“Suitability of a new alignment correction method for industrial CT”, iCT Conference 2014, ISSN 1435-4934, Vol. 19(6), pages 245 to 252, is a paper of Matthias Elter et al. discussing exact knowledge of the geometrical configuration of an industrial CT scanner which is essential for high quality CT image reconstruction. Geometrical misalignment can result in severe misalignment artifacts like blurring and the loss of spatial resolution. In computed tomography, redundantly measured rays (i.e. multiple measurements of a line integral through an object) pose a computationally efficient possibility to quantify the rawdata quality in a cost function and thus to reduce many kinds of artifacts. A general downside of such a cost function is that the portion of redundant rays is generally small and depends on the specific data acquisition geometry. The suitability of using information associated to plane integrals instead of line integrals approach for industrial CT applications is evaluated and proven.

The publication “Epipolar Consistency in Transmission Imaging” of André Aichert et al., IEEE, pages 1 to 15, concerns the optimization of data consistency. This paper presents the derivation of the Epipolar Consistency Conditions (ECC) between two X-ray images from the Beer-Lambert law of X-ray attenuation and the Epipolar Geometry of two pinhole cameras, using Grangeat's theorem. The authors motivate the use of oriented projective geometry to express redundant line integrals in projection images and define a consistency metric, which can be used, for instance, to estimate patient motion directly from a set of X-ray images. The authors describe in detail the mathematical tools to implement an algorithm to compute the Epipolar Consistency metric and investigate its properties with detailed random studies on both artificial and real FD-CT data. A set of 6 reference projections of the CT scan of a fish were used to evaluate accuracy and precision of compensating for random disturbances of the ground truth projection matrix using an optimization of the consistency metric. In addition, the authors use three X-ray images of a pumpkin to prove applicability to real data. The authors conclude, that the metric might have potential in applications related to the estimation of projection geometry. By expression of redundancy between two arbitrary projection views, the authors in fact support any device or acquisition trajectory which uses a cone-beam geometry. The authors discuss certain geometric situations, where the ECC provide the ability to correct 3D motion, without the need for 3D reconstruction.

However, there is also another type of misalignment, neither purely global nor purely local. It is a misalignment that undergoes small changes from one projection images to another of the sequence, incrementally increasing or decreasing in one or some geometric parameters. Examples of this type of misalignment are drifts in the X-ray focal spot, drifts in an object location or orientation, or continuous motion patterns of a scanned patient. These misalignments are called “drifting geometric misalignments” and can accumulate incrementally. Up to now, the way of coping with a projection data set that includes drifting geometric misalignments is to directly apply conventional jitter reduction on this projection data set.

### SUMMARY

The inventor is aware of the following conventional jitter reduction method:
receiving the projection data set, wherein the projection data set comprises a sequence of projection images (I1, I2, I3, . . . IN) and a sequence of geometry information (P1, P2, P3, . . . PN), wherein each geometry information (P1, P2, P3, . . . PN) is associated with the respective projection image (I1, I2, I3, . . .IN),
processing all projection images (I1, I2, I3, . . . IN) iteratively image-by-image in an arbitrary order of the sequence, wherein the processing includes
calculating a cost function C(IA, PA) for the current projection image (Ik) being processed depending on all projection images (I1, I2, I3, . . . IN) and associated geometry information (P1, P2, P3, . . . PN), where k is the index of the current projection image (Ik) being processed,
modifying the geometry information (Pk) associated with the current projection image (Ik) to numerically minimize the cost function,
repeating the calculating step and modifying step the current projection image (Ik) until a stopping criterium is met,
reconstructing a 3D image data set on basis of the projection images (I1, I2, I3, . . . IN) and the associated modified geometry information (P1, P2, P3, . . . PN) after the processing step.

This approach produces regularly good outcomes in many scenarios. However, there are cases of drifting geometric misalignments when such conventional jitter reduction method completely fails. In these cases, no misalignment might be detected at all, even though notable misalignment patterns do exist. Therefore, jitter reduction yields insufficient 3D image data sets in such cases. One reason is that the degrees of freedom in misalignment estimation in above conventional jitter reduction method are too large causing not a stable and robust performance in cases of drifting geometric misalignments.

From other approaches it is known that it is possible to implement compensation methods that explicitly incorporate motion models of drifting geometric misalignments. This is, for example, done in cardiac computed tomography (CT) or C-arm CT, where periodic (drifting) motions are present. Also, for Dental cone beam CT or orthopedic 3D imaging, it is typical to involve such motion models that are depending on human joints and/or motion ranges. Apparently, such motion models are application specific. It is difficult to develop a well-generalized motion model that performs well in some or even all relevant types of applications, such as in medical as well as industrial 3D X-ray imaging. Furthermore, using an explicit motion model can result in a significant change in the misalignment compensation algorithm, which is not wanted by the inventor.

An underlying technical problem of one or more example embodiments of the present invention is to provide a better computer-implemented method for handling drifting geometric misalignments in a projection data set of 3D X-ray imaging for reconstructing a 3D image data set, a better data processing unit, a better 3D X-ray imaging system, a better computer program product and a better computer-readable medium wherein in particular the image quality of the reconstructed 3D image data set is improved.

At least this problem is solved by the features of the independent claims. Advantageous embodiments are disclosed within the dependent claims.

One or more example embodiments of the present invention relate, in one aspect, to a computer-implemented method for handling drifting geometric misalignments in a projection data set of 3D X-ray imaging for reconstructing a 3D image data set, the method comprising:
receiving the projection data set, wherein the projection data set comprises a sequence of projection images (I1, I2, I3, . . . IN) and a sequence of geometry information (P1, P2, P3, . . . PN), wherein each geometry information (P1, P2, P3, . . . PN) is associated with the respective projection image (I1, I2, I3, . . . IN),
processing all projection images (I2, I3, . . . IN) except the first projection image iteratively image-by-image in the order of the sequence, wherein the processing includes
calculating a cost function C(IA, PA) for the current projection image (Ik) being processed depending on those projection images (I1, I2, I3, . . . Ik) and associated geometry information (P1, P2, P3, . . . Pk) in the sequence up to and including the current projection image (Ik), where A={1, . . . k} and k is the index of the current projection image (Ik) being processed,
modifying the geometry information (Pk) associated with the current projection image (Ik) to numerically minimize the cost function,
repeating the calculating step and modifying step the current projection image (Ik) until a stopping criterium is met,
reconstructing a 3D image data set on basis of the projection images (I1, I2, I3, . . . IN) and the associated modified geometry information (P1, P2, P3, . . . PN) after the processing step.

One or more example embodiments of the present invention relate, in one aspect, to a computer-implemented method for handling drifting geometric misalignments in a projection data set of 3D X-ray imaging for reconstructing a 3D image data set, the computer-implemented method comprising:
receiving the projection data set, wherein the projection data set includes a sequence of projection images and a sequence of geometry information, wherein each geometry information is associated with a respective projection image;
processing projection images in the sequence of projection images, except a first projection image, iteratively image-by-image in order of the sequence of projection images, wherein the processing includes
   calculating a cost function for a current projection image being processed depending on projection images and associated geometry information in the sequence of projection images up to and including the current projection image,
   modifying geometry information associated with the current projection image to numerically minimize the cost function, and
   repeating the calculating and the modifying for the current projection image until a stopping criterium is met; and
reconstructing a 3D image data set based on the projection images and the associated modified geometry information after the processing.

One or more example embodiments of the present invention relate in one aspect to a data processing unit, comprising a processor and

a memory comprising instructions stored thereon which, when executed by the processor, cause the data processing unit to perform the method according to one or more example embodiments of the present invention.

One or more example embodiments of the present invention relate, in one aspect, to a 3D X-ray imaging system, comprising the data processing unit, according to one or more example embodiments of the present invention, an X-ray source for emitting X-rays, an X-ray detector for acquiring X-rays having passed an object, wherein the data processing unit is configured to receive the projection data set which is based on the detected X-rays. The 3D X-ray imaging system can be configured for cone-beam computed tomography. The 3D X-ray imaging system is in particular configured for medical imaging and/or industrial imaging applications. The 3D X-ray imaging system can be a medical computed tomography (CT) imaging system, a C-arm CT imaging system, a cardiac CT imaging system, a dental CT imaging system, an orthopedic CT imaging system, an industrial CT imaging system, a non-destructive testing CT imaging system, and/or a customs inspections CT imaging system.

The X-ray source may comprise a thermionic emitter or a cold emitter. There could be also several X-ray sources. The X-ray source or the X-ray sources could be mounted movable around the object or spatially fixed to the patient. The X-rays detector could be mounted movable around the object or could be arranged spatially fixed to the patient.

One or more example embodiments of the present invention relate, in one aspect, to a computer program product, comprising a computer program, the computer program being loadable into a memory unit of a data processing unit, including program code sections to make the data processing unit execute the method according to one or more example embodiments of the present invention when the computer program is executed in said data processing unit.

One or more example embodiments of the present invention relate, in one aspect, to a non-transitory computer-readable medium, on which program code sections of a computer program are saved, said program code sections being loadable into and/or executable in a data processing unit to make the data processing unit execute the method according to one or more example embodiments of the present invention when the program code sections are executed in said data processing unit.

One or more advantages of one or more example embodiments of the present invention are as follows:

One or more example embodiments of the present invention yield a beneficial modification of a well-established, theoretically sound and practically successful, conventional jitter reduction method. One or more example embodiments of the present invention advance the conventional method into being capable of robustly estimating and compensating for drifting geometric misalignments.

One difference between the method, according to one or more example embodiments of the present invention, and the conventional method is in the calculation of the cost function that is, according to one or more example embodiments of the present invention, optimized in order to improve geometry information for a given 3D X-ray projection data set. In the conventional method, the cost function is defined to use all data as input, and therefore it measures the consistency of the entire projection data set. That is, the cost function of the known solution involves several projection images which have not yet been processed during the cost function minimization process. The equation of the cost function is independent of the index of the current projection image that is currently optimized. In other words, this equation is constant during the entire processing step.

In the method, according to one or more example embodiments of the present invention, the equation of the cost function varies during the processing step image-by-image: it (only) involves data with a lower or equal projection index with reference to the current projection image that is currently optimized. The cost function of the proposed solution depends in particular on data which has been already optimized. The geometry information of the currently processed projection image is therefore made consistent with the previous projection images, disregarding any data that might come after the current projection image in indices bigger than k. The method, according to one or more example embodiments of the present invention, is in particular handling drifting geometric misalignment incrementally. This makes the optimization beneficially more robust. In evaluations based on simulated data or real data, the method, according to one or more example embodiments of the present invention, clearly outperforms the conventional method, in particular, in scenarios of drifting geometric misalignments.

Another advantage is that the method, according to one or more example embodiments of the present invention, requires minor implementation changes over the conventional method. In particular, the need to apply explicit major changes to the cost function (such as additional terms that enforce slowly changing parameters in the motion patterns) does not exist. Complexity that would come with these explicit changes is advantageously avoided.

The computer-implemented method, according to one or more example embodiments of the present invention, is in particular executable via the data processing unit. The computer-implemented method is in particular configured for reconstructing the 3D image data set.

Handling drifting geometric misalignments means in particular reducing drifting geometric misalignments and/or optimizing image quality. The drifting geometric misalignments can comprise at least one of drifts in X-ray focal spot position, in object location of the object of the projection data set, in object orientation of the object, or in continuous motion patterns of the object. Drifting geometric misalignments occur in particular over time and/or during the sequence.

The projection data set of 3D X-ray imaging is in particular a projection data set acquired via a 3D X-ray imaging system. The projection data set comprises in particular projection images of the object allowing a reconstruction of the 3D image data set of the object. The object can in particular be a patient or a device or a material. 3D X-ray imaging can be medical imaging and/or industrial imaging.

The projection data set comprises in particular a plurality of projection images and a plurality of associated geometry information. The projection data set comprises in particular a number of at least three projection images and an equal number of associated geometry information. The number can be higher than three, for example, more or equal than 20, or preferably more or equal than 100. The number is typically as high that the 3D image data set is reconstructed on basis of projection images acquired over scan projection angles covering at least 180° of the object.

The projection images are typically 2D X-ray images. A projection image comprises in particular an attenuation profile or attenuation contour resulting from absorption and/or attenuation of emitted X-rays within the object. The attenuation profile or attenuation contour reflects in particular the density distribution of the object. A projection image is typically based on the X-rays detected by the X-ray detector. It is possible that the X-ray detector is configured to generate a projection image on basis on the detected X-rays. Additionally or alternatively, the data processing unit can be configured to generate a projection image on basis on the detected X-rays. The detected X-rays are typically converted into X-ray signals prior to their conversion into a projection image. The projection images can be buffered and/or stored in a memory, e.g. of the 3D X-ray imaging system or the data processing unit or a network-based storage system,

The projection images are typically acquired subsequently and therefore typically vary in their projection acquisition time. It is an option that some or all projection images are typically acquired at the same time. The projection acquisition time of a projection image is defined with reference to the time when the emitted X-rays pass the object and/or are detected by the X-ray detector.

The projection images are typically acquired from different scan projection angles and optionally at different longitudinal positions of the object. The difference between respective scan projection angels is ideally constant. Alternatively, the differences between at least three projection images may vary due to the different type of misalignments.

The geometry information of the respective projection images varies typically at least due to the different respective scan projection angle. The geometry information defines mathematically and/or geometrically the position from where the X-rays represented in the individual projection image are emitted and/or where those X-rays are detected. Associated means, that by definition a projection image and the respective geometry information are linked. Sorting or resorting the projection images doesn't break up their associations with the respective geometry information. That is, by resorting the projection images automatically the associated geometry information is also resorted and vice versa.

Typically, each geometry information can be either determined prior to, during, or after acquiring the respective projection images. The determining can comprise sensing, measuring, recognizing, and/or calculating the geometry information. Additionally or alternatively, the geometry information can be buffered and/or stored in the memory and/or loaded from such memory.

The projection data set is in particular received at an interface of the data processing unit. The interface can be wired or wireless. The projection data set can be transferred, for example, from the memory via a network connecting the memory or the 3D X-ray imaging system with the data processing unit and/or via other data connections.

The sequence of projection images is typically in the same sequence as the geometry information. That is, at the processing of the current projection image in particular the associated geometry information and not any other arbitrary geometry information is used.

The processing occurs typically via the processor of the data processing unit. The processor can be of any type of regular processor as known in the art, including physical processors and/or virtualized processors and/or cloud-based processors and/or a combination thereof.

The processing step loops over all projection images except the first projection image. Having N projection images, the processing step is executed at least N-1 times. The processing step is executed iteratively, that is, repeated for the current projection image until the stopping criterium is met. The calculating step and modifying step are executed at least N-1 times, regularly much more often, but not with the first projection image as current projection image.

The processing step is executed image-by-image in a series. That is, not two projection images are processed in parallel.

The processing step is executed in the order of the sequence. That is, the projection images as ordered in the sequence at the beginning are processed one-by-another without a random selection of the upcoming current projection image.

For example, the cost function of the second projection image depends on the first projection image and its associated geometry information, and the second projection image and its associated geometry information. The cost function of the third projection image depends on the first projection image and its associated geometry information, the second projection image and its associated geometry information, and the third projection image and its associated geometry information.

The processing step includes for each projecting image being processed at least two steps: the modifying step and the calculating step. Each of both steps can comprise one or several partial steps to be executed.

The calculating the cost function occurs generally as known in the art, however, including the input values, according to one or more example embodiments of the present invention. The cost function in particular quantifies inconsistency between those projection images and associated geometry information in the sequence up to and including the current projection image. The cost function is in particular computed from values of the projection images and/or can be based on measuring deviations of redundant data in the projection domain. The lower the value of the cost function is, the better is the data consistency and the lower is the inconsistency. Good consistency means that the geometry information advantageously describes accurately the points of view from where the projection images have been acquired.

The modifying the geometry information associated with the current projection image to numerically minimize the cost function occurs generally as known in the art. Minimizing the cost functions means in particular optimizing the cost function. The modifying the geometry information associated with the current projection image occurs for instance based on some practical numerical optimization routines, until an updated geometry information for the current projection image is found that leads towards a minimization of the cost function. The modified geometry information replaces the previous geometry information associated with the current projection image.

Repeating the calculating step and modifying step means that instead of the previous geometry information associated with the current projection image before the modifying, for the next minimization cost function calculation the modified geometry information associated with the current projection image and the remaining previous input values are used for calculating the cost function.

One stopping criterium is that the cost function value is below a certain threshold. Another stopping criterium is that a counter of the repetition reaches a specific limit or that a time limit for the processing of the current projection image would be exceeded unless the repetition is stopped. The stopping criterium function is typically evaluated and/or executed prior to starting a new round of repeating the calculating and modifying step.

The modifying the geometry information may comprise modifying a parameter of the geometry information pre-selected by a user, wherein the parameter is selected and received prior to the processing. In particular the user of the 3D X-ray imaging system and/or the data processing unit may select the parameter via an input device of the 3D X-ray imaging system and/or an input device of the data processing unit. One or more parameters may be pre-selected by the user. It is possible that the modifying the geometry information is restricted to modifying only one or more parameters pre-selected by the user such that other parameters which are not pre-selected cannot be modified. The modifying the parameter comprises particular an updating of the parameter.

The modifying the geometry information may comprise modifying the value of the parameter within a limit pre-defined by the user, wherein the limit is defined and received prior to the processing. In particular the user of the 3D X-ray imaging system and/or the data processing unit may define the limit via an input device of the 3D X-ray imaging system and/or an input device of the data processing unit. One or more limits may be pre-defined by the user. It is possible, that the modifying the geometry information is restricted to modifying a value of one or more parameters only within the limit pre-defined by the user. The modifying a value of the parameter comprises particular an updating of the value within the limit of the parameter.

Ideally, the input device and/or the 3D X-ray imaging system are configured to transfer the pre-selected parameter and/or the pre-defined limit to the data processing unit prior to the processing step. The user-definition and user-selection are in particular beneficial if only those parameters are selected and/or those limits are defined which are the ones where a misalignment is likely to occur.

After N-1 projection images have been processed typically the 3D image data set is reconstructed. The reconstructing the 3D image data set on basis of the projection images and the associated modified geometry information generally occurs as known in the art. The reconstructed 3D image data set may be provided via a display device and/or stored in the memory. Advantageously, the reconstructed 3D image data set comprises a high quality, preferably sharp, representation of the object.

One embodiment of the present invention relates in one aspect to that the sequence of projection images and the sequence of geometry information are resorted prior to the processing. The resorting may occur by the scan projection angle or the projection acquisition time or an ECG information or a temperature information.

The data processing unit can be a computer device or a computer network device or a cloud computing device or a handheld device. The data processing unit can be realized as a data processing system or as a part of a data processing system. The data processing system can, for example, comprise cloud-computing system, a computer network, a computer, a tablet computer, a smartphone or the like. The data processing system can comprise hardware and/or software. The hardware can be, for example, a processor system, a memory system and combinations thereof. The hardware can be configurable by the software and/or be operable by the software.

The computer program product can be, for example, a computer program or comprise another element apart from the computer program. This other element can be hardware, for example a memory device, on which the computer program is stored, a hardware key for using the computer program and the like, and/or software, for example a documentation or a software key for using the computer program.

Reference is made to the fact that the described methods and the described data processing unit as well as the described device are merely preferred example embodiments of the present invention and that the present invention can be varied by a person skilled in the art, without departing from the scope of the present invention provided it is specified by the claims. Said features, advantages or alternative embodiments of the apparatus apply also to the method and vice-versa.

Independent of the grammatical term usage, individuals with male, female or other gender identities are included within the term.

### BRIEF DESCRIPTION OF THE DRAWINGS

The present invention will be illustrated below with reference to the accompanying figures using example embodiments. The illustration in the figures is schematic and highly simplified and not necessarily to scale.

FIG. 1 shows a conventional jitter reduction method,

FIG. 2 shows a method according to one or more example embodiments of the present invention,

FIG. 3 shows the method according to one or more example embodiments of the present invention in another figure and

FIG. 4 shows a first exemplary embodiment of the method.

FIG. 5 shows a schematic representation of a medical imaging system including a data processing device, according to one or more example embodiments of the present invention.

### DETAILED DESCRIPTION

FIG. 1 shows a schematic view of a conventional jitter reduction method.

The conventional jitter reduction method comprises the steps:
receiving the projection data set, wherein the projection data set comprises a sequence of projection images I1, I2, I3, . . . IN and a sequence of geometry information P1, P2, P3, . . . PN, wherein each geometry information P1, P2, P3, . . . PN is associated with the respective projection image I1, I2, I3, . . . IN,
processing all projection images I1, I2, I3, . . . IN iteratively image-by-image in an arbitrary order of the sequence, wherein the processing includes
calculating a cost function C(IA, PA) for the current projection image Ik being processed depending on all projection images I1, I2, I3, . . . IN and associated geometry information P1, P2, P3, . . . PN, where k is the index of the current projection image Ik being processed,
modifying the geometry information Pk associated with the current projection image Ik to numerically minimize the cost function,
repeating the calculating step and modifying step the current projection image Ik until a stopping criterium is met,
reconstructing a 3D image data set on basis of the projection images I1, I2, I3, . . . IN and the associated modified geometry information P1, P2, P3, . . . PN after the processing step.

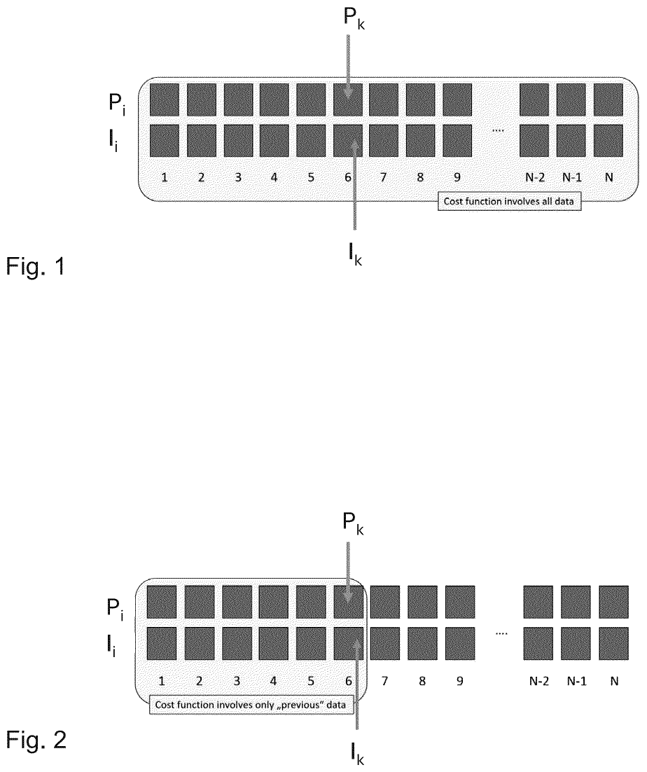

FIG. 2 shows a schematic view of a method according to one or more example embodiments of the present invention.

The computer-implemented method for handling drifting geometric misalignments in a projection data set of 3D X-ray imaging for reconstructing a 3D image data set, the method comprises the steps:
receiving the projection data set, wherein the projection data set comprises a sequence of projection images I1, I2, I3, . . . IN and a sequence of geometry information P1, P2, P3, . . . PN, wherein each geometry information P1, P2, P3, . . . PN is associated with the respective projection image I1, I2, I3, . . . IN,
processing all projection images I2, I3, . . . IN except the first projection image iteratively image-by-image in the order of the sequence, wherein the processing includes
calculating a cost function C(IA, PA) for the current projection image Ik being processed depending on those projection images I1, I2, I3, . . . Ik and associated geometry information P1, P2, P3, . . . Pk in the sequence up to and including the current projection image Ik, where A={1, . . . k} and k is the index of the current projection image Ik being processed,
modifying the geometry information Pk associated with the current projection image Ik to numerically minimize the cost function,
repeating the calculating step and modifying step the current projection image Ik until a stopping criterium is met,
reconstructing a 3D image data set on basis of the projection images I1, I2, I3, . . . IN and the associated modified geometry information P1, P2, P3, . . . PN after the processing step

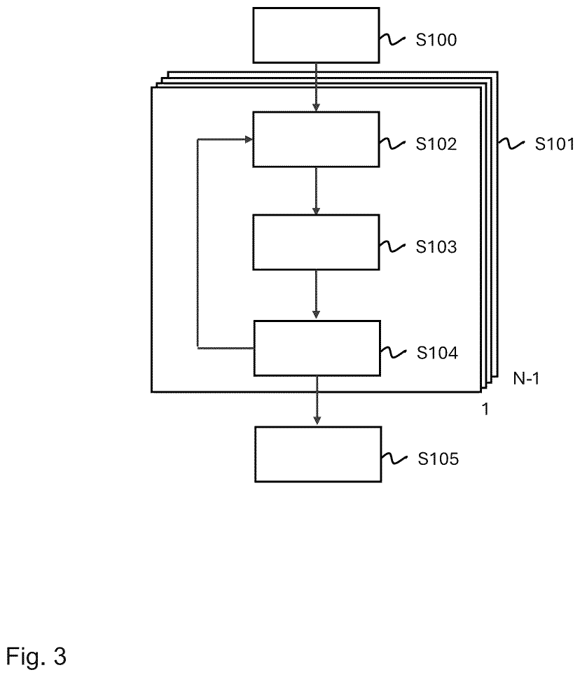

FIG. 3 shows a flow diagram of the method according to one or more example embodiments of the present invention.

Step S100 denotes receiving the projection data set, wherein the projection data set comprises a sequence of projection images I1, I2, I3, . . . IN and a sequence of geometry information P1, P2, P3, . . . PN, wherein each geometry information P1, P2, P3, . . . PN is associated with the respective projection image I1, I2, I3, . . . IN.

Step S101 denotes processing all projection images I2, I3, . . . IN except the first projection image iteratively image-by-image in the order of the sequence. That is, step S101 is executed N-1 times.

Step S102 denotes that the processing includes calculating a cost function C(IA, PA) for the current projection image Ik being processed depending on those projection images I1, I2, I3, . . . Ik and associated geometry information P1, P2, P3, . . . Pk in the sequence up to and including the current projection image Ik, where A={1, . . . k} and k is the index of the current projection image Ik being processed. The cost function C(IA, PA) quantifies inconsistency between those projection images I1, I2, I3, . . . Ik and associated geometry information P1, P2, P3, . . . Pk in the sequence up to and including the current projection image. The cost function C(IA, PA) is based on measuring deviations of redundant data in the projection domain.

Step S103 denotes that the processing further includes modifying the geometry information Pk associated with the current projection image Ik to numerically minimize the cost function.

Step S104 denotes that the processing further includes repeating the calculating step and modifying step the current projection image Ik until a stopping criterium is met.

Step S105 denotes reconstructing a 3D image data set on basis of the projection images I1, I2, I3, . . . IN and the associated modified geometry information P1, P2, P3, . . . PN after the processing step.

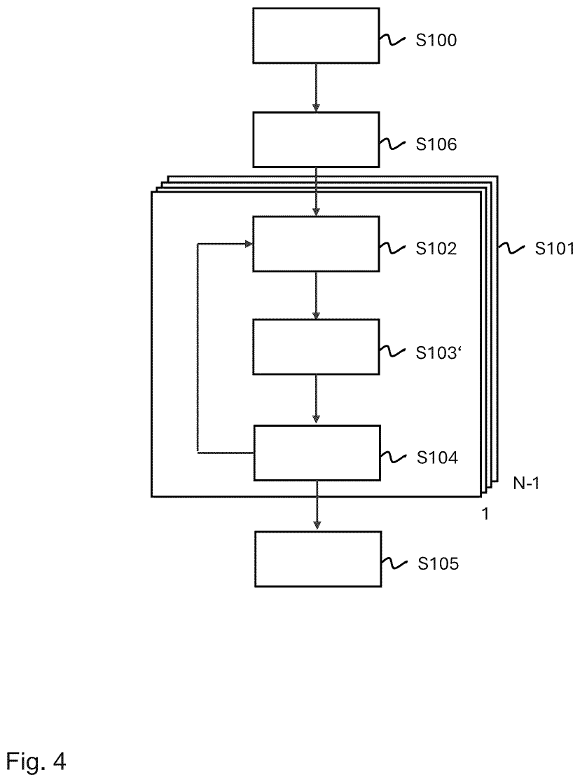

FIG. 4 shows a flow diagram of the first exemplary embodiment of the method.

Step S106 denotes that the sequence of projection images I1, I2, I3, . . . IN and the sequence of geometry information P1, P2, P3, . . . PN are resorted prior to the processing. The resorting may occur by the scan projection angle or the projection acquisition time or by an ECG information or a temperature information.

Step S103′ denotes that the processing further includes modifying the geometry information Pk associated with the current projection image Ik to numerically minimize the cost function. The modifying the geometry information P1, P2, P3, . . . Pk comprises modifying a parameter of the geometry information P1, P2, P3, . . . Pk pre-selected by a user, wherein the parameter is selected and received prior to the processing. The modifying the geometry information P1, P2, P3, . . . Pk comprises modifying the value of the parameter within a limit pre-defined by the user, wherein the limit is defined and received prior to the processing.

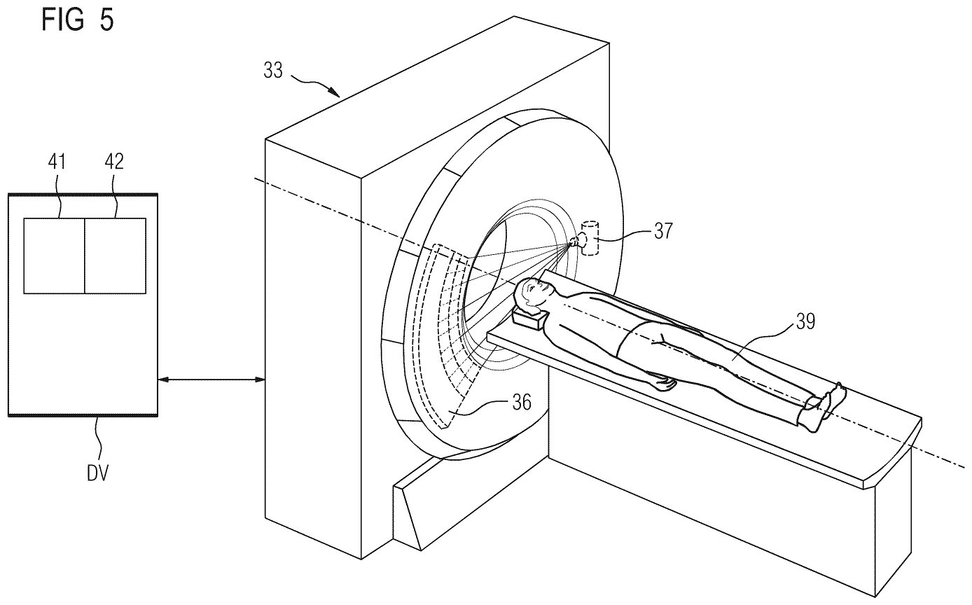

FIG. 5 shows a schematic representation of a 3D X-ray imaging system including a data processing device, according to one or more example embodiments of the present invention.

Referring to FIG. 5, the 3D X-ray imaging system 33 includes an X-ray source 37, an X-ray detector 36, and a data processing unit DV. The X-ray source 37 is configured to emit X-rays and the X-ray detector 36 is configured to detect X-rays having passed through an object 39. The data processing unit DV is configured to receive the projection data set which is based on detected X-rays.

The data processing unit DV includes a processor 41 and a memory 42. The memory 42 stores instructions, wherein the instructions, when executed by the processor 41, cause the data processing unit DV to perform a computer-implemented method according to one or more example embodiments described herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been illustrated and described in detail by the preferred embodiments, the present invention is not limited to the disclosed examples and other variations can be derived by a person skilled in the art without departing from the scope of the present invention.

What is claimed is:

1. A computer-implemented method for handling drifting geometric misalignments in a projection data set of 3D X-ray imaging for reconstructing a 3D image data set, the computer-implemented method comprising:
receiving the projection data set, wherein the projection data set includes a sequence of projection images and a sequence of geometry information, wherein each geometry information is associated with a respective projection image;
processing projection images in the sequence of projection images, except a first projection image, iteratively image-by-image in order of the sequence of projection images, wherein the processing includes
calculating a cost function for a current projection image being processed depending on projection images and associated geometry information in the sequence of projection images up to and including the current projection image,
modifying geometry information associated with the current projection image to numerically minimize the cost function, and
repeating the calculating and the modifying for the current projection image until a stopping criterium is met; and
reconstructing a 3D image data set based on the projection images and the associated modified geometry information after the processing.

2. The computer-implemented method as claimed in claim 1, wherein the sequence of projection images and the sequence of geometry information are sorted prior to the processing.

3. The computer-implemented method as claimed in claim 2, wherein the sequence of projection images and the sequence of geometry information are sorted by scan projection angle or projection acquisition time.

4. The computer-implemented method as claimed in claim 2, wherein the sequence of projection images and the sequence of geometry information are sorted by ECG information or temperature information.

5. The computer-implemented method as claimed in claim 1, wherein modifying of the geometry information comprises:
modifying a parameter of geometry information selected by a user, wherein
the parameter is selected and received prior to the processing.

6. The computer-implemented method as claimed in claim 5, wherein modifying of the geometry information comprises:
modifying a value of the parameter within a limit defined by the user, wherein
the limit is defined and received prior to the processing.

7. The computer-implemented method as claimed in claim 1, wherein the cost function quantifies inconsistency between the projection images and associated geometry information in the sequence of projection images up to and including the current projection image.

8. The computer-implemented method as claimed in claim 1, wherein the cost function is based on measuring deviations of redundant data in a projection domain.

9. The computer-implemented method as claimed in claim 1, wherein the drifting geometric misalignments include at least one of drifts in X-ray focal spot position, in object location of an object of the projection data set, in object orientation of the object, or in continuous motion patterns of the object.

10. A data processing unit, comprising:
a processor, and
a memory including instructions stored thereon, wherein the instructions, when executed by the processor, cause the data processing unit to perform the computer-implemented method of claim 1.

11. A 3D X-ray imaging system, comprising:
the data processing unit of claim 10,
an X-ray source configured to emit X-rays, and
an X-ray detector configured to detect X-rays having passed through an object, wherein
the data processing unit is configured to receive the projection data set which is based on detected X-rays.

12. A non-transitory computer program product comprising a computer program, the computer program being loadable into a memory of a data processing unit, and the computer program including program code sections to make the data processing unit execute the computer-implemented method of claim 1 when the computer program is executed at said data processing unit.

13. A non-transitory computer-readable medium storing computer-executable instructions that, when executed at a data processing unit, cause the data processing unit to perform the computer-implemented method of claim 1.

14. The computer-implemented method as claimed in claim 3, wherein modifying of the geometry information comprises:
modifying a parameter of geometry information selected by a user, wherein
the parameter is selected and received prior to the processing.

15. The computer-implemented method as claimed in claim 4, wherein modifying of the geometry information comprises:
modifying a parameter of geometry information selected by a user, wherein
the parameter is selected and received prior to the processing.

16. The computer-implemented method as claimed in claim 3, wherein the cost function quantifies inconsistency between the projection images and associated geometry information in the sequence of projection images up to and including the current projection image.

17. The computer-implemented method as claimed in claim 4, wherein the cost function quantifies inconsistency between the projection images and associated geometry information in the sequence of projection images up to and including the current projection image.

18. The computer-implemented method as claimed in claim 3, wherein the cost function is based on measuring deviations of redundant data in a projection domain.

19. The computer-implemented method as claimed in claim 3, wherein the drifting geometric misalignments include at least one of drifts in X-ray focal spot position, in object location of an object of the projection data set, in object orientation of the object, or in continuous motion patterns of the object.

20. The computer-implemented method as claimed in claim 4, wherein the drifting geometric misalignments include at least one of drifts in X-ray focal spot position, in object location of an object of the projection data set, in object orientation of the object, or in continuous motion patterns of the object.
